(19) United States
(12) Patent Application Publication
(10) Pub. No.: US 2004/0015152 A1
(43) Pub. Date: Jan. 22, 2004

(54) Reducing torque needed to perform a cardiovascular procedure

(76) Inventor: Ronald W. Day, Salt Lake City, UT (US)

Correspondence Address: KIRTON & McCONKIE, Suite 1800, 60 East South Temple, Salt Lake City, UT, 84111, US

(21) Appl. No.: 10/441,918

(22) Filed: May 20, 2003

Related U.S. Application Data
(60) Provisional application No. 60/381,982, filed on May 20, 2002.

(51) Int. Cl. A61B 6/00

(52) U.S. Cl. 604/532; 600/435; 600/585

(57) ABSTRACT

Systems and methods for providing a medical device for use in right coronary arteriography or right coronary angioplasty and stent placement, wherein the medial device and use thereof reduces the amount of torque required to perform the cardiovascular procedure. A catheter is provided that is U-shaped and includes an angle near the distal end. The catheter is used to perform a selective right coronary arteriography procedure at or near the orifice of a blood vessel with a limited amount of torque and catheter manipulation. The safety and efficacy of the right coronary arteriography procedure using the limited-torque technique and catheter results in a decreased incidence of intimal injury due to a reduction in the amount of torque required to finalize proper placement of the catheter.

### RELATED APPLICATIONS

This application claims priority to U.S. Provisional Patent Application Serial No. 60/381,982 filed May 20, 2002, entitled DESIGN OF A CATHETER FOR RIGHT CORONARY ARTERIOGRAPHY AND A GUIDING CATHETER FOR RIGHT CORONARY ANGIOPLASTY AND STENT PLACEMENT AND LIMITED TORQUE METHODS OF USE.

### BACKGROUND OF THE INVENTION

1. Field of the Invention

The present invention relates to reducing torque needed to perform a cardiovascular procedure. In particular, the present invention relates to systems and methods for providing a medical device for use in right coronary arteriography or right coronary angioplasty and stent placement, wherein the medial device and use thereof reduces the amount of torque required to perform the cardiovascular procedure.

2. Background and Related Art

A variety of medical procedures are currently performed on the cardiovascular system of a patient. For example, selective coronary arteriography, which is an X-ray examination of the blood vessels or chambers of the heart, is occasionally indicated in patients with congenital or acquired heart disease. In the procedure, a catheter is inserted into a blood vessel of the patient's groin or arm. The tip of the catheter is positioned either in the heart or at the beginning of the arteries supplying the heart. A contrast medium is injected through the catheter that is visible by X-ray.

While this procedure has proven to be beneficial, problems exist. For example, typical right coronary catheters are not appropriately sized for patients due to the patient's age or variations in aortic and coronary anatomy. Further, a typical catheter requires torque to appropriately position the tip of the catheter within the orifice of the blood vessel. The torque can project the catheter deeper than necessary within the blood vessel. Moreover, the torque can force the catheter against the luminal wall of the blood vessel and cause a coronary spasm or intimal injury. Such problems compromise the safety and efficacy of the medical procedure.

A study performed on the methods and results of right coronary arteriography occurring during a sample period of time demonstrates the frequency of intimal damage incurred by patients. In the study, a standard-torque technique was associated with a focal region of proximal coronary artery spasm in eight patients, a nonsustained ventricular tachycardia in one patient, and ST segment changes in one patient. These complications were associated with the use of a right Judkins coronary catheter in nine patients and a right Amplatz coronary catheter in one patient. The episode of ventricular tachycardia occurred when contrast was manually injected with the catheter tip inadvertently positioned within a conal branch. These complications occurred in patients with a median age of 12 years and a range of 1 to 17 years. Of the eight patients who experienced proximal coronary spasm, seven have had a right coronary arteriography procedure repeated.

Thus, while techniques currently exist that allow medical procedures to be performed on the cardiovascular system of a patient, challenges still exist. Accordingly, it would be an improvement in the art to augment or even replace current techniques with other techniques.

### SUMMARY OF THE INVENTION

The present invention relates to reducing torque needed to perform a cardiovascular procedure. In particular, the present invention relates to systems and methods for providing a medical device for use in right coronary arteriography or right coronary angioplasty and stent placement, wherein the medial device and use thereof reduces the amount of torque required to perform the cardiovascular procedure.

Implementation of the present invention takes place in association with a catheter that is selectively used to perform a medical procedure on the cardiovascular system of a patient. In at least one implementation, the catheter is shaped to perform a selective right coronary arteriography procedure at or near the orifice of a blood vessel with a limited amount of torque and catheter manipulation. The safety and efficacy of the right coronary arteriography procedure using the limited-torque technique and apparatus of the present invention results in a decreased incidence of intimal injury.

In at least one implemenatation, the shape of the catheter for right coronary arteriography is generally U-shaped and includes an angle near the distal end that places the soft distal tip closer to the orifice of the blood vessel. Accordingly, there is a reduction in the amount of torque required to finalize proper placement of the catheter.

While the methods and processes of the present invention have proven to be particularly useful in the area of performing a right coronary arteriography, those skilled in the art will appreciate that the methods, apparatuses and/or processes of the present invention can be used in a variety of different applications and in a variety of different medical procedures. Examples of such medical procedures include right coronary arteriography, angioplasty, stent placement, etc.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a side view of a representative embodiment of the present invention for use in association with performing a medical procedure on a patent;

FIG. 2A illustrates a front view of the representative embodiment of FIG. 1, wherein the distal end is aligned with the proximal end;

FIG. 2B illustrates a front view of the representative embodiment of FIG. 1, wherein the distal end is transverse from the proximal end; and

FIG. 3 illustrates a representative placement of the embodiment of FIG. 1 into a blood vessel of a patient.

### DETAILED DESCRIPTION OF THE INVENTION

The present invention relates to reducing torque needed to perform a cardiovascular procedure. In particular, the present invention relates to systems and methods for providing a medical device for use in right coronary arteriography or right coronary angioplasty and stent placement, wherein the medial device and use thereof reduces the amount of torque required to perform the cardiovascular procedure.

Embodiments of the present invention take place in association with a catheter that is selectively used to perform a medical procedure on the cardiovascular system of a patient. The cardiovascular system of a patient includes the patient's heart and blood vessels.

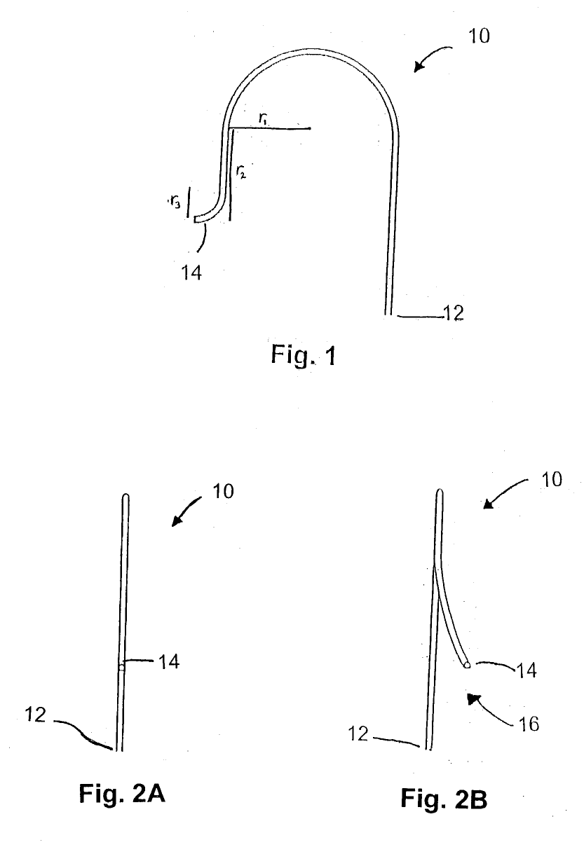

With reference now to FIG. 1, a side view of a representative catheter of the present invention is illustrated. In FIG. 1, catheter 10 is provided for use in association with performing a medical procedure on a patent. For example, catheter 10 includes proximal end 12 and distal end 14, and is shaped to perform a selective right coronary arteriography procedure at or near the orifice of a blood vessel with a limited amount of torque and catheter manipulation, as will be further discussed below.

A coronary arteriography procedure is an X-ray examination of the blood vessels or chambers of a patient's heart. A catheter (e.g., catheter 10) is typically inserted into a blood vessel in the patient's groin or arm. The distal end 14 of catheter 10 is positioned either in the heart or at the beginning of the arteries supplying the heart. A contrast medium is injected into the blood stream and is visible by X-ray. The coronary arteriography procedure is used to help physicians evaluate, for example, the number and severity of blockages in arteries that supply blood to the heart.

In FIG. 1, the shape of catheter 10 for performing a medical procedure (e.g., right coronary arteriography) on the cardiovascular system of a patient is generally U-shaped and includes an angle near distal end 14 that places the soft distal tip of the catheter closer to the orifice of the blood vessel. Accordingly, there is a reduction in the amount of torque required to finalize proper placement of the catheter. As such, the safety and efficacy of the right coronary arteriography procedure using a representative limited-torque technique and apparatus embraced by embodiments of the present invention results in a decreased incidence of intimal injury to the patient.

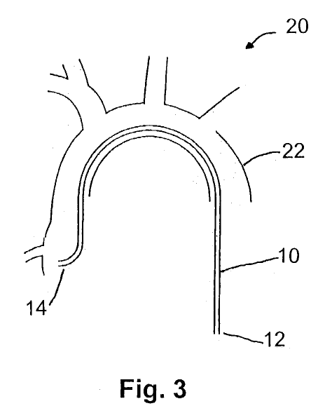

As illustrated in FIG. 1, catheter 10 includes an elongated tubular member that is curved in three dimensions so that the distal end 14 may be selectively placed in proximity with a right coronary ostium of a patient with only a minimal amount of torque exerted in the tubular body. The curvature of the tubular member includes multiple radii. A first radius (illustrated as r₁) corresponds to the general U-shaped curvature of the tubular body. A second radius (illustrated as r₂) corresponds to placing the distal end 14 in a transverse position from the proximal end 12, wherein the transverse position is more clearly illustrated in FIG. 2, and will be further discussed below. A third radius (illustrated as r₃) corresponds to the angling away of the tip of the distal end 14 from the proximal end 12.

A reduction in the incidence of injuries is enabled through the representative configuration of catheter 10. To further discuss the representative configuration of catheter 10, reference is now made to FIGS. 2A-2B. In FIG. 2A, a front view illustration of catheter 10 is provided, wherein distal end 14 is aligned with proximal end 12. Accordingly, in the embodiment of FIG. 2A, the catheter 10 includes the above-referenced first radius (illustrated as r₁, which corresponds to the general U-shaped curvature of the tubular body) and the above-referenced third radius (illustrated as r₃, which corresponds to the angling away of the tip of the distal end 14 from the proximal end 12), which are illustrated in FIG. 1.

In a further embodiment, as illustrated in FIG. 2B, distal end 14 of catheter 10 is transverse from proximal end 12. Accordingly, the embodiment illustrated in FIG. 2B includes the above-referenced first radius (illustrated as r₁, which corresponds to the general U-shaped curvature of the tubular body), the above-referenced second radius (illustrated as r₂, which corresponds to proving the distal end 14 of catheter 10 in a transverse position from the proximal end 12), and the above-referenced third radius (illustrated as r₃, which corresponds to the angling away of the tip of the distal end 14 from the proximal end 12), which are illustrated in FIG. 1. In FIG. 2B, the transverse orientation of distal end 14 with proximal end 12 is illustrated by separation 16.

In accordance with embodiments of the present invention, the catheter (e.g., catheter 10 of FIG. 2A or FIG. 2B) reduces the amount of torque needed to position the catheter within or near the orifice of the blood vessel. This technique can be performed safely and effectively in pediatric or adult patients. Another benefit of a catheter in accordance with the present invention and its use is that the origin of the right coronary artery and the distribution of its branches can clearly be defined with minimal risk of complications when the limited-torque technique is used appropriately.

With reference now to FIG. 3, placement 20 illustrates that the configuration of catheter 10 allows catheter 10 to be selectively positioned over, for example, the aortic arch 22 of a patient with the distal end 14 of catheter 10 pointing anteriorly near the orifice of the right coronary artery. Accordingly, catheter 10 can be positioned within or near the orifice of the blood vessel with a limited amount of torque and manipulation in patients of different age.

Accordingly, embodiments of the present invention embrace a method for performing a medical diagnosis or treatment of the right coronary artery of a patient, wherein the method includes the steps of providing a catheter having an elongated hollow body with a proximal end portion and a distal end portion, wherein the elongated hollow body is substantially curved in three dimensions so that the distal end portion may be selectively placed in proximity with a right coronary ostium of a patient with only a minimal amount of torque exerted on the tubular member; and advancing the distal end portion through the descending aorta, over the aortic arch and into the ascending aorta, wherein the advancement of the catheter over the aortic arch locates distal end portion in proximity with the right coronary ostium of the patient.

In accordance with embodiments of the present invention, the indications for selective coronary arteriography may vary for children, young adults and older patients. The proximal segment of the right coronary artery is to be imaged clearly to identify anomalous coronary branches, hypoplasia of the coronary orifice, or proximal coronary artery stenosis in selected patients. Proximal coronary artery stenosis is a particular concern in patients with Kawasaki disease, supravalvar aortic stenosis and surgical interventions which involve the transfer or reimplantation of the coronary arteries. Proximal coronary abnormalities may be overlooked if torque projects the catheter tip deeper than desired within the vessel using the standard-torque technique. An aortogram may also fail to demonstrate proximal abnormalities when contrast fills a sinus and obscures the origin and proximal portion of a vessel.

Coronary spasm is not always a result of catheter irritation in adults with coronary artery disease. In each case of coronary spasm, a focal region of narrowing corresponded with the position of the catheter tip. It is unknown whether infants and children are less likely than adults to experience diffuse coronary spasm during angiography. It is possible that the small vessels of pediatric patients are more vulnerable to catheter-mediated spasm and injury. The limited-torque technique has a potential advantage in defining the proximal and distal portions of the right coronary artery with the catheter tip simply positioned at, or near, the orifice of the vessel. Thereby, the safety of selective coronary arteriography may be improved by avoiding injury to the delicate coronary arteries of infants and children. This may also be important in the setting of Kawasaki disease where the standard-torque technique may increase the risk of myocardial ischemia by disrupting thrombus within a proximal coronary aneurysm.

In accordance with embodiments of the present invention, the safety and efficacy of selective right coronary arteriography may be improved by the new catheter design and technique which requires a limited amount of torque to position the catheter within or near the orifice of the vessel.

While the methods and processes of the present invention have proven to be particularly useful in the area of performing a right coronary arteriography, those skilled in the art will appreciate that the methods, apparatuses and/or processes of the present invention can be used in a variety of different applications and in a variety of different medical procedures. Examples of such medical procedures include right coronary arteriography, angioplasty, stent placement, etc.

Thus, as discussed herein, the embodiments of the present invention embrace a reduction of torque needed to perform a cardiovascular procedure. In particular, the present invention relates to systems and methods for providing a medical device for use in right coronary arteriography or right coronary angioplasty and stent placement, wherein the medial device and use thereof reduces the amount of torque required to perform the cardiovascular procedure. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A right coronary catheter for use in performing a medical procedure on a cardiovascular system of a patient, the catheter comprising:
an elongated tubular member having a proximal end and a distal end, the tubular member being curved in three dimensions so that the distal end is selectively placed in proximity with a right coronary ostium of a patient with only a minimal amount of torque exerted on the tubular member.

2. A right coronary catheter as recited in claim 1, wherein the medical procedure is a coronary arteriography procedure.

3. A right coronary catheter as recited in claim 1, wherein the medical procedure is an angioplasty and stent placement procedure.

4. A right coronary catheter as recited in claim 1, wherein a first portion of the tubular member corresponds to a first radius that provides a U-shaped configuration in the tubular member.

5. A right coronary catheter as recited in claim 4, wherein a second portion of the tubular member corresponds to a second radius that provides the distal end of the tubular member angled away from the proximal end of the tubular member.

6. A right coronary catheter as recited in claim 5, wherein a third portion of the tubular member corresponds to a third radius that provides the distal end of the tubular member in a transverse position in relation to the proximal end.

7. A right coronary catheter as recited in claim 6, wherein the third portion of the tubular member includes the second portion of the tubular member.

8. A right coronary catheter as recited in claim 1, wherein the tubular member includes a soft distal tip.

9. A method for performing a medical diagnosis or treatment of the right coronary artery of a patient, the method comprising steps of:
providing a catheter having an elongated hollow body with a proximal end portion and a distal end portion, wherein the elongated hollow body is substantially curved in three dimensions so that the distal end portion may be selectively placed in proximity with a right coronary ostium of a patient with only a minimal amount of torque exerted on the tubular member; and
advancing the distal end portion through the descending aorta, over the aortic arch and into the ascending aorta, wherein the advancement of the catheter over the aortic arch locates distal end portion in proximity with the right coronary ostium of the patient.

10. The method as recited in claim 9, further comprising a step of using the catheter to perform a coronary arteriography procedure.

11. The method as recited in claim 9, further comprising a step of using the catheter to perform an angioplasty and stent placement procedure.

12. The method as recited in claim 9, wherein the catheter includes a U-shaped configuration.

13. The method as recited in claim 9, wherein the proximal end portion of the catheter is transverse from the distal end portion.

14. The method as recited in claim 8, wherein the distal end portion of the catheter angles away from the proximal end portion.

15. A right coronary catheter for use in performing a medical procedure on a cardiovascular system of a patient, the catheter comprising:
an elongated tubular member having a proximal end and a distal end, the tubular member being curved in two dimensions so that the distal end is selectively placed in proximity with a right coronary ostium of a patient with only a minimal amount of torque exerted on the tubular member.

16. A right coronary catheter as recited in claim 15, wherein the medical procedure is one of:
(i) a coronary arteriography procedure; and
(ii) an angioplasty and stent placement procedure.

17. A right coronary catheter as recited in claim 15, wherein a first portion of the tubular member corresponds to a first radius that provides a U-shaped configuration in the tubular member.

18. A right coronary catheter as recited in claim 17, wherein a second portion of the tubular member corresponds to a second radius that provides the distal end of the tubular member angled away from the proximal end of the tubular member.

19. A right coronary catheter as recited in claim 19, wherein the third portion of the tubular member includes the second portion of the tubular member.
